(19) United States
(12) Patent Application Publication
(10) Pub. No.: US 2002/0132266 A1
(43) Pub. Date: Sep. 19, 2002

(54) Method for modifying and identifying functional sites in proteins

(76) Inventors: Leodevico L. Ilag, Munich (DE); Jocelyn H. Ng, Munich (DE)

Correspondence Address: CLARK & ELBING LLP, 101 FEDERAL STREET, BOSTON, MA, 02110, US

(21) Appl. No.: 10/142,125

(22) Filed: May 9, 2002

Related U.S. Application Data
(63) Continuation-in-part of application No. 09/856,285, filed on Aug. 28, 2001.
Application No. 09/856,285 is a 371 of international application No. PCT/EP99/09052, filed on Nov. 23, 1999.

(30) Foreign Application Priority Data
Nov. 24, 1998 (DE) DE 19854196.1

(51) Int. Cl. C12Q 1/68; G01N 33/53; G01N 33/542; G01N 33/537; G01N 33/543

(52) U.S. Cl. 435/6; 435/7.1; 435/7.9; 436/518

(57) ABSTRACT

The invention relates to a method for identifying one or more functional sites in a protein, which is characterized in that

a) the target protein is contacted with a binding partner linked to a laser-activatable marker (tag), (BP-tag), to form a complex of target protein and BP-tag,

b) the complex of target protein and BP-tag is irradiated with laser light to generate free radicals which selectively alter the bound target protein at the binding site, and

c) the selectively altered region of the protein is identified by a combination of protein cleavage and mass spectrometry. The invention further relates to an apparatus for carrying out the method according to the invention.

### CROSS-REFERENCES TO RELATED APPLICATIONS

This application is a Continuation-in-Part of U.S. patent application Ser. No. 09/856,285, filed Aug. 08, 2001, which is the International Application No. EP99/09052, filed Nov. 23, 1999, which claims benefit of German Patent Application 19854196.1, filed Nov. 24, 1998, now German Patent DE 19854196, which are incorporated by reference herein in their entireties.

### SUMMARY OF THE INVENTION

The invention relates to a method for the specific modification and identification of functional sites in a target protein. A correlation can be made between the specific amino acid(s) and their function in the protein or their function as ligand binding sites through the loss/gain of function of the protein and subsequent determination of the modified amino acids (epitope mapping). The method is based on combined use of chromophore-assisted laser inactivation (CALI) and mass spectrometry or protein sequencing. The invention also relates to an apparatus for carrying out this method.

### BACKGROUND OF THE INVENTION

The techniques of recombinant DNA and of protein preparation make deeper understanding of the relations between protein structure and protein function easier. Methods of random mutagenesis, specific genetic selection and screening methods make it possible rapidly to gain information at the protein level, but have the disadvantage that particular functional domains of the protein are very tolerant to amino acid substitutions, that some mutagenesis reagents preferentially attack particular DNA sequences, and possible mutations are limited. The information provided by these methods is thus limited.

The alternative of site-specific mutagenesis methods provides a more direct and more specific way of identifying amino acids which are important for the biological function, such as, for example, the alanine scanning mutagenesis method. In this method there is systematic mutation of each amino acid to alanine, and the function is determined. Each loss of function is related to the specific amino acid. This technique can be applied to proteins without the relevant three-dimensional structure and without unknown functional domains. In addition, this entails in each case modification of only one amino acid, which is why specific functions involving a plurality of amino acids are not detected. In addition, the method becomes more complicated as the molecular size increases. This is a disadvantage for analysing the function of gene products in connection with diseases because these are often large proteins containing multiple structural domains.

Furthermore, other recombinant DNA techniques have been used for epitope mapping and for mapping ligand binding sites, in which there is expression of random antigen fragments with mutations introduced at the DNA level (J. Immunol. Meth., 141, 245-252, 1991, J. Immunol. Meth., 180, 53-61, 1995, Virology, 198, 346-349, 1994). Recently, the phage display of random peptide banks have been used for epitope mapping (J. Immunol., 153, 724-729, 1994, PNAS, 93, 1997-2001, 1996).

Another variant of epitope mapping of proteins is the rapid automatic synthesis of selected peptides (J. Endocrinol., 145, 169-174, 1995) and the use of combinatorial peptide banks (FEBS Letters, 352, 167-170, 1994). Although these methods are reliable for linear epitopes, they have been unsuccessful with nonlinear or discontinuous epitopes. The variant of using overlapping peptides was also unsatisfactory with discontinuous epitopes. This entails screening individual peptides or peptide mixures for binding to the ligand by means of ELISA, with free and bound peptides competing for the ligand (for example antibody). However, this method is elaborate and time-consuming.

Another approach used a combination of protein modification and mass spectrometry (Anal. Biochem., 196, 120-125, 1991). This entails the ligand being bound to the receptor, and the complex being modified with acetic anhydride, resulting in acetylation of the lysine residues. The proteolytic cleavage mixtures of the two proteins are analysed by mass spectrometry and compared with the corresponding fragments from the untreated complex. The modified lysines can easily be detected, these lysines not being protected in the complex, and the unmodified lysines forming part of the interaction between ligand and receptor. This technique is thus confined to interactions involving lysine residues. Another variant is based on differential proteolysis (Protein Science, 4, 1088-1099, 1995), with sites sensitive to proteolysis becoming protease-resistant after complex formation.

There has also been a description of identification by mass spectrometry of a proteolytic cleavage of free peptide antigen comparing with the pattern from the peptide antigen bound to an antibody. The identification took place by ²⁵²Cf plasma desorption mass spectrometry (PNAS 87, 9848-9852, 1990). There has furthermore been a description of a combination of immunoprecipitation and matrix-assisted laser desorption mass spectrometry (MALDI-MS) (PNAS, 93, 4020-4024, 1996). This entails an antigenic protein being cleaved into smaller fragments and precipitated with an antibody of interest. The immunoprecipitated peptides are identified by MALDI-MS, and the antibody-binding region is determined. In this method there was also separation of proteolytically cleaved peptides by affinity capillary electrophoresis and identification by electrospray mass spectrometry (ACE-MS) (Anal. Chem., 69, 3008-3014, 1997). Injection of the peptide mixture is followed by injection of the antibody. Peptides which bind to the antibody are trapped and therefore do not migrate. The bound peptide is investigated by the subtraction screening method in order to determine the epitope residue on the peptide. However, this technique is confined to linear epitopes and cannot be applied to discontinuous epitopes.

### DESCRIPTION OF DRAWINGS

FIG. 1 Diagrammatic depiction of the identification of functional sites in proteins

FIG. 2: Apparatus for carrying out CALI

FIG. 3: Serine-protease activity of thrombin, measured with Chromozym TH as substrate. Activity in U/ml for thrombin alone (left bars), with specific aptamer THR15 (middle bars) and with fluorescein-labeled aptamer Flu-THR15 (right bars).

FIG. 4: NanoES-MS analysis of isolated thrombin heavy chain (MW 32345 Da). A: THR alone, not irradiated; B: THR irradiated in the presence of free fluorescein; C: THR irradiated in the presence of fluorescein-labeled aptamer, Flu-THR15.

FIG. 5: NanoES-MS analysis of the peptide mixtures obtained by hydrolysis with trypsin of isolated thrombin heavy chain. The 3 panels correspond to the same samples as in FIG. 4 after digestion with trypsin. A: THR alone, not irradiated; B: THR irradiated in the presence of free fluorescein; C: THR irradiated in the presence of fluorescein-labeled aptamer, Flu-THR15.

### DETAILED DESCRIPTION OF THE INVENTION

The present invention is therefore based on the object of overcoming the problems of the prior art mentioned. The intention is to provide a method with which any functional sites in any proteins can be identified. It is intended preferably to be able to identify sites involved in a ligand interaction, and epitopes. In particular, the method according to the invention should be applicable to nonlinear and discontinuous epitopes and without knowledge of the three-dimensional structure of a protein. It is also intended according to the invention for determination of the protein function to be possible without inactivating the molecule. It is additionally intended that the method be simple to use, quickly carried out and automatable. It is further intended to provide an apparatus for carrying out the method according to the invention simply.

This object is achieved by a method for identifying one or more functional sites in a protein, which is characterized in that

a) the target protein is contacted with a binding partner linked to a laser-activatable marker (tag), (BP-tag), to form a complex of target protein and BP-tag,

b) the complex of target protein and BP-tag is irradiated with laser light to generate free radicals which selectively alter the bound target protein at the binding site, and

c) the selectively altered region of the protein is identified by a combination of protein cleavage and mass spectrometry,

and by an apparatus for carrying out the method.

It has been found, surprisingly, that combination of the CALI technique with mass spectrometry permits reliable and rapid identification of functional sites on proteins. According to the invention, a target protein is modified by CALI to inactivate it. Subsequently, the modified region of the protein is determined by mass spectrometry. Tandem MS and/or de novo sequencing are preferably used. This makes it possible in a simple way to make a correlation between the amino acid and its biological function. This correlation between structure and function allows information to be gained for example about the correlation between a particular metabolic activity and the corresponding site in the molecule, about proteins with pathological alterations, cancer-promoting proteins etc. It is thus also possible specifically to inactivate unwanted (for example pathological) proteins.

The target protein is initially contacted with a binding partner under conditions such that complex formation takes place, but the proteins are not denatured. Ideally, the conditions correspond to the physiological conditions of the cellular environment of the proteins. The binding partner is linked to a laser-activatable marker. The laser-activatable marker can be any marker suitable for covalent or noncovalent linkage to a binding partner, i.e. an amino acid sequence, and can be activated so that it is able to generate free radicals. The marker is preferably activated with laser light, but activation is also possible by peroxidases (hydrogen peroxide/horseradish peroxidase system). Examples of such markers are AMCA-S, AMCA, BODIPY and variants thereof, Cascade Blue, Cl-NERF, dansyl, dialkylaminocoumarin, 4′,5′-dichloro-2′,7′-dimethoxy fluorescein, DM-NERF, eosin, eosin F3S, erythrosin, hydroxycoumarin, Isosulfan Blue, lissamine rhodamine B, malachite green, methoxycoumarin, naphthofluorescein, NBD, Oregon Green 488, 500, 514, PyMPO, pyrene, Rhodamine 6G, Rhodamine Green, Rhodamine Red, Rhodol Green, 2′,4′,5′,7′-tetrabromosulphonefluorescein, tetramethylrhodamine, Texas Red or X-rhodamine. The marker is preferably malachite green isothiocyanate, fluorescein isothiocyanate or 4′,5′-bis(1,3,2-dithioarsolan-2-yl)fluorescein. The irradiation takes place with laser light of a wavelength which is absorbed by the particular chromophore.

The binding partner can be any binding partner for the appropriate protein. It is preferably scfv, Fab, a diabody, an immunoglobulin-like molecule, a peptide, RNA, DNA, PNA or a small organic molecule.

The binding partner is preferably derived from a combinatorial bank. This can be any combinatorial bank, for example protein bank, peptide bank, cDNA bank, mRNA bank, bank with organic molecules, scfv bank with immunoglobulin superfamily, protein display bank etc. The following can be presented in the banks: all types of proteins, for example structural proteins, enzymes, receptors, ligands, all types of peptides including modifications, DNAs, RNAs, combinations of DNAs and RNAs, hybrids of peptides and RNA or DNA, all types of organic molecules, for example steroids, alkaloids, natural substances, synthetic substances etc. The presentation can take place in various ways, for example as phage display system (for example filamentous phages such as M13, fl, fd etc., lambda phage display, viral display etc), presentation on bacterial surfaces, ribosomes etc.

Using the CALI technique, target proteins are directly and specifically inactivated (cf. PNAS, 85, 5454-5458, 1988; Trends in Cell Biology, 6, 442-445, 1996). CALI can be used to convert binding reagents such as antibodies or other ligands into function-blocking molecules. This entails a binding partner (BP) being linked for example to the dye malachite green (MG). On irradiation with laser light of a wavelength which is not significantly absorbed by the cellular components, this dye generates free radicals. For the determination, the BP linked to MG (=BP−MG) is incubated with the protein sample of interest. The region to be inactivated is selected and irradiated with a laser beam at 620 nm. This light is absorbed by MG to produce short-lived free radicals which selectively inactivate the proteins bound to the BP-MG in a radius of 15 Å through irreversible chemical modification. This system can be used for in vitro and in vivo assays and for intra- and extracellular target molecules.

The laser wavelength used for irradiation has to be adapted appropriately, depending on the dye which is linked to the BP. The dye has also an impact on the mechanismn of inactivation, e.g. due to the nature of the free radicals produced during irradiation. Different species of free radicals are able to diffuse various distances. This can further lead to modifications in the vicinity of the binding site of the protein, depending on the diffusion radius of the free radical.

A protein inactivated in this way is then cleaved. It is possible to use for this purpose a protease which cleaves specifically after a residue, for example Lys-C, Glu-C, Asp-N. Examples thereof are trypsin, chymotrypsin, papain etc. Chemical cleavage is also possible, for example with cyanogen bromide (specific for Met), 3-bromo-3-methyl-2-(2-nitrophenylmercapto)-3H-indole (BNPS-skatole; specific for Trp), 2-nitro-5-thiocyanatobenzoic acid (specific for Cys) and Fe-EDTA.

It is then possible to evaluate the modified amino acids by mass spectrometry and comparison with the untreated target protein.

Very recent developments in mass spectrometry have led to rapid identification of proteins (PNAS USA, 93, 14440-14445, 1996). As soon as the target protein has been inactivated by CALI it is possible to identify the modified amino acids of the inactivated protein which are presumably responsible for the inactivation by mass spectrometry and, where appropriate, de novo sequencing (Rapid Commun. Mass Spectrom., 11, 1015-1024, 1997; Rapid Commun. Mass Spectrom., 11, 1067-1075, 1997).

The identity of the proteins complexed according to the invention with a binding partner is established by a combination of protein cleavage and mass spectrometry, where appropriate de novo sequencing.

This entails a protein which has been treated in this way and is provided with a marker initially being separated from the target protein by an electrophoresis or by a chromatography. The isolated and modified protein is then cleaved either chemically or proteolytically by the methods described above. This can take place either in the gel (that is to say by direct elution of the target protein from the gel after the separation, followed by subsequent protein separation) or in solution. Methods for cleavage in the gel are known and described, for example, in Advanced Methods in Biological Mass Spectrometry, EMBLLaboratory, Heidelberg, 1997 or in Shevchenko, A., et al., Anal. Chem. 68:850-858, 1996.

The MALDI analysis is carried out in a manner known per se.

It is necessary for the nanoelectrospray analysis (nanoES) to extract the tryptic peptides from the pieces of gel. To do this, the pieces of gel are washed successively with ammonium bicarbonate, acetonitrile, dilute formic acid and again with acetonitrile. The supernatants are combined and dried in a vacuum centrifuge. The sample is dissolved in 80% strength formic acid, rapidly diluted with water and then desalted.

The analysis by mass spectrometry can be carried out in various ways known per se, for example using an ionization source such as electrospray (Chapman, J. R., et al., Methods in Molecular biology, 61, JR Chapman editor, Humana Press Inv. Totowa N.J., USA, 1996) including nanoelectrospray (Wilm. M. and Mann, M., Anal. Chem. 68, 1-8, 1996) and matrix-assisted laser desorption and ionization (MALDI) (Siuzdak, G. Mass Spectrometry for Biotechnology, Academic Press Inc. 1996) or using a combination of mass analysers such as quadrupole, time of flight, magnetic sector, Fourier transformation ion cyclotron resonance and quadrupole ion capture.

If the peptides in the cleavage mixture are insufficient for unambiguous establishment of the identity of the investigated site in the protein, further sequence information can be obtained by further fragmentation in the mass spectrometer such as, for example, by decomposition downstream of the source in MALDI-TOF, MS/MS (tandem mass spectrometry), MSⁿ. The proteins can additionally be identified by de novo sequencing.

Since in CALI there is photochemical modification of certain amino acids of the target protein, the MS can be used in two stages to identify which amino acids have been modified in what way. The method can be carried out with low and high resolution.

At low resolution it is firstly possible to determine by peptide mass mapping (Anal. Chem., 69, 4741-4750, 1997; Biochem. Soc. Transactions, 24, 893-896, 1996; Anal. Chem., 69, 1706-1714, 1997) which segments of the protein have been modified. In the second stage, at higher resolution, tandem mass spectrometry and/or de novo sequencing on selected peptides can be used to determine the site of the modifications. It is possible with a mass spectrometer configuration able to resolve masses from 0 up to 0.03 dalton (Rapid Commun. Mass Spectrom., 11, 1015-1024) (qQTOF and other mass analysers) to determine which specific amino acids have been modified by CALI; it is even possible to determine the type of modification from the incremental mass gain or loss.

The free radicals generated by CALI lead to a modification of the oxidation-sensitive amino acid side chains such as His, Met, Cys and Trp. There may also be modification of other amino acid side chains. This results in significant changes in mass, because oxygen has been added on. Comparison of the peptide fragments after the cleavage between CALI-treated fragments and untreated sample leads to an approximate localization of the modified amino acids. These amino acids are identified more accurately by de novo sequencing.

It is unnecessary to know the exact nature of the modification. The method according to the invention permits the modification to be recognized from the difference between the treated and untreated samples. The advantage of this method is that it can be used to elucidate discontinuous epitopes even if the three-dimensional structure of the protein is unknown.

The modifications induced by CALI can also be elucidated by using other methods such as parent ion scans (J. Mass Spectrom, 32, 94-98, 1997), which increases the speed of analysis. This detects only the peptides altered by CALI.

If insufficient information is obtainable because of limited peptide fragmentation or doubtful assignment of mass, it is possible to carry out a de novo mass sequencing (Rapid Commun. Mass Spectrom., 11, 1015-1024, 1997). In this method, the peptides are labelled with 180. This is done by carrying out the tryptic cleavage in the gel with a cleavage buffer which contains 50% (vol/vol) H₂¹⁸O purified by microdistillation. A QqTOF mass spectrometer is preferably used. Clear results are possible by reading the doublet owing to the 1:1 ¹⁶O/¹⁸O ratio. The mass difference of the doublet is an indicator of the charge state of the specific peptide. Using different charge states it is possible to read off up to 15 amino acids in a sequence for a given peptide. Comparison of the ¹⁸O-labelled spectra of the untreated and CALI-modified proteins provides unambiguous information about the modified amino acids.

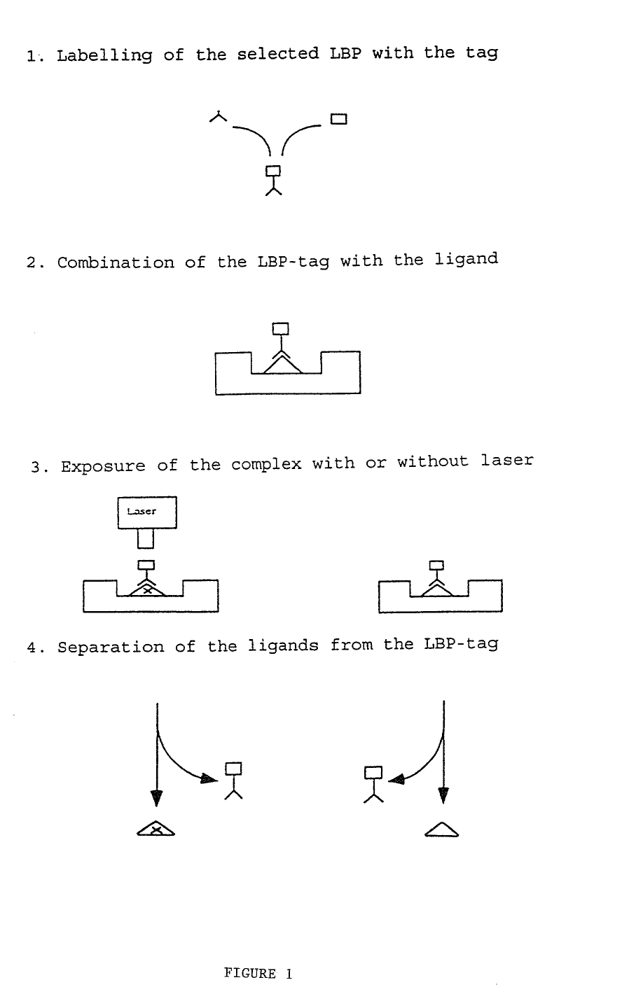

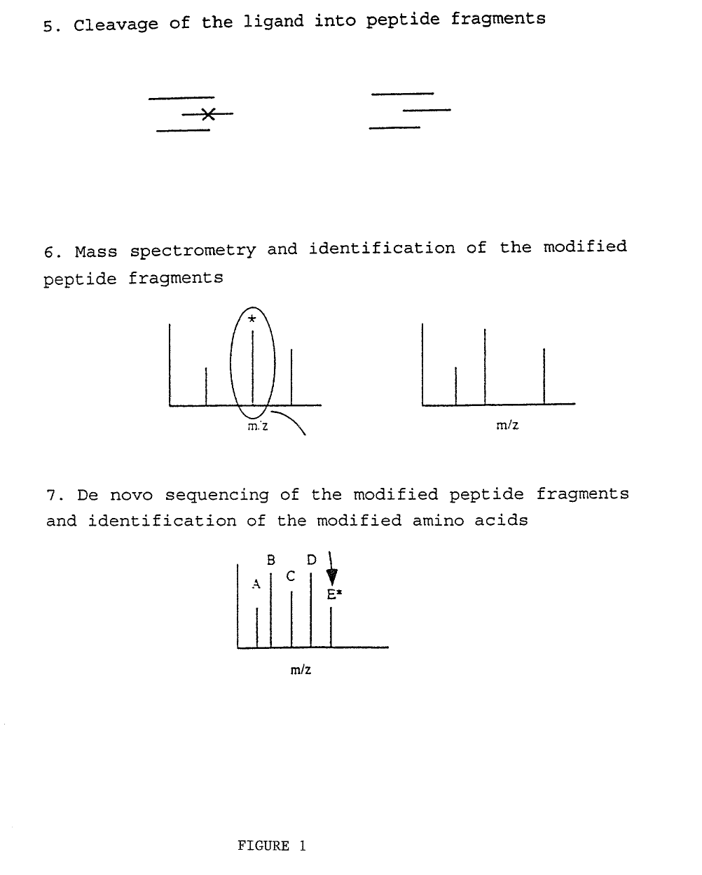

FIG. 1 shows the scheme for identifying functional sites in proteins.

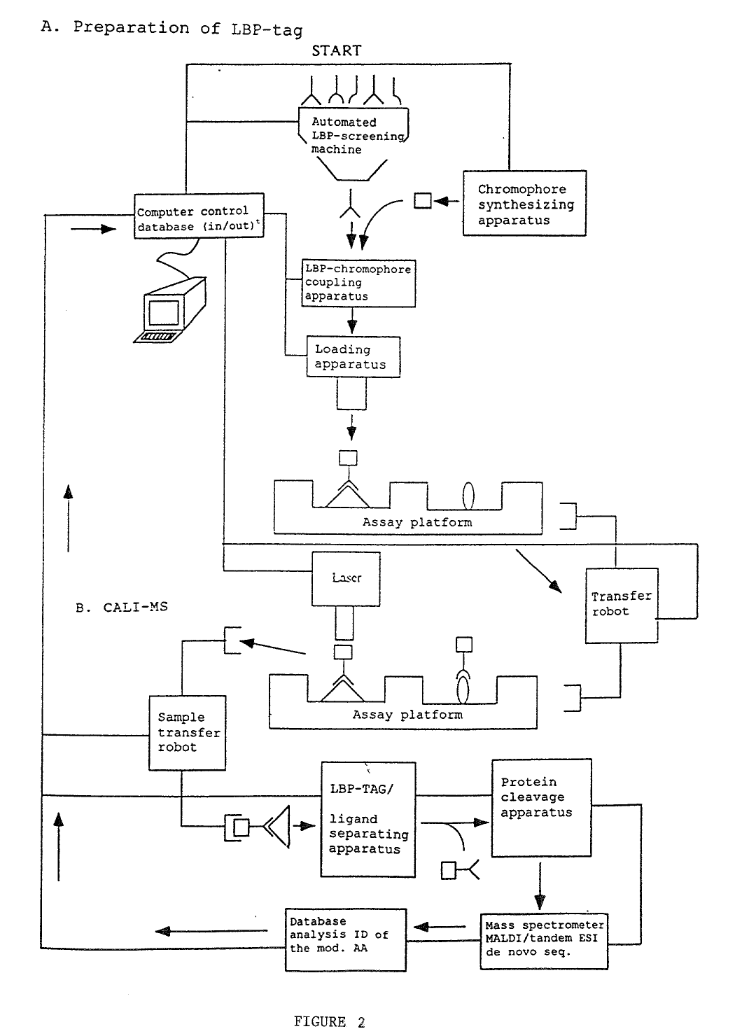

The invention also relates to an apparatus for carrying out the method according to the invention. This is shown in FIG. 2. This apparatus is an automated system of integrated independent units/parts which can be used for identifying functional sites in proteins. Part A relates to the preparation of the LBP-tag. An automated LBP screening machine reveals specific LBPs directed against specific target molecules/ligands, while the chromophore synthesis appratus produces chromophores of choice. The selected LBPs and the synthesized chromophores are chemically linked in an LBP-chromophore coupling apparatus, resulting in the LBP-tag. This LBP-tag is transferred into a loading apparatus which transfers the LBP-tab into predetermined cavities coated with the target molecule/ligand in the assay platform.

A transfer robot then moves the assay platform into the laser system in order to initiate the second part B. The samples are irradiated with the laser at the required wavelength in order to induce a modification by free radicals. The irradiated samples are then transferred by a sample transfer robot into an LBT-tag/ligand separating apparatus in order to isolate the ligand. The ligand is then cleaved in a protein cleavage apparatus. The peptide fragments are then analysed with a mass spectrometer in order to detect changes in mass, or in order to carry out a direct sequencing. Data from the mass spectra are then used for the analysis in the data base, which finally leads to identification of the amino acids or peptide fragments. All parts of the apparatus are connected to a central computer system for control and analysis.

In order that the invention described may be more fully understood, the following examples are set forth. It should be understood that these examples are for illustrative purposes only and are not to be construed as limiting this invention in any matter.

### EXPERIMENTAL PART

### Example 1

### EPITOPE Mapping of Thrombin By Cali-MS

Reagents

A fluorescein-labeled, thrombin-specific aptamer (15-mer oligonucleotide with the sequence GGTTGGTGTGGTTGG with the fluorescein moiety linked to its 5′ end, Metabion GmbH, Martinsried, Germany), which inhibits clotting activity (Bock et al., 1992, Nature 355, 564-566) was used. Purified human α-Thrombin was a kind gift from Prof. Bode of the MPI in Martinsried. Fluorescein isothiocyanate (FITC) was purchased from Molecular Probes (Eugene, Oreg.), all other reagents used were of the highest purity available.

Complex Preparation

Complexes were formed by mixing 7.6 μM thrombin with the fluorescein-labeled aptamer (1:2 molar ratio) in 50 mM Tris buffer, pH 8.3, 100 mM NaCl, and an incubation time of 30 min on ice in the dark.

Control samples were prepared as described above, either omitting the aptamer, using a non-labeled aptamer or an equal concentration of FITC. Subsequently, the solutions were diluted 20-fold with the same buffer. The final concentrations were 0.38 μM thrombin and 0.76 μM aptamer. The samples were pipetted in 100 μl aliquots into wells of a flat bottom 96-well microtiter plate (Nunc A/S, Roskilde, DK), typically 1.2 ml or 12 wells total per sample. The plate was blocked with 1% BSA in PBS for 1 hour and then washed extensively prior to use.

Mesurement of Serine-Protease Activity

The serine-protease activity of thrombin was measured using Chromozym TH (Roche Diagnostics GmbH, Mannheim, Germany) as substrate. The protocol provided by the manifacturer was used: 920 μl of Tris buffer 50 mM, pH 8.3, NaCl 200 mM and containing 0.1% BSA were mixed in a plastic cuvette with 50 μl of Chromozym 1.9 mM in water and equilibrated at room temperature before addition of 30 μl of the 0.38 μM thrombin solution. The absorbance increase at 405 nm, due to the cleavage of Chromozym TH into a residual peptide and free 4-aniline by thrombin, was followed with the time. The absorbance difference per minute, calculated in the linear range, was used to calculate the thrombin activity in U/ml.

Cali Experiements

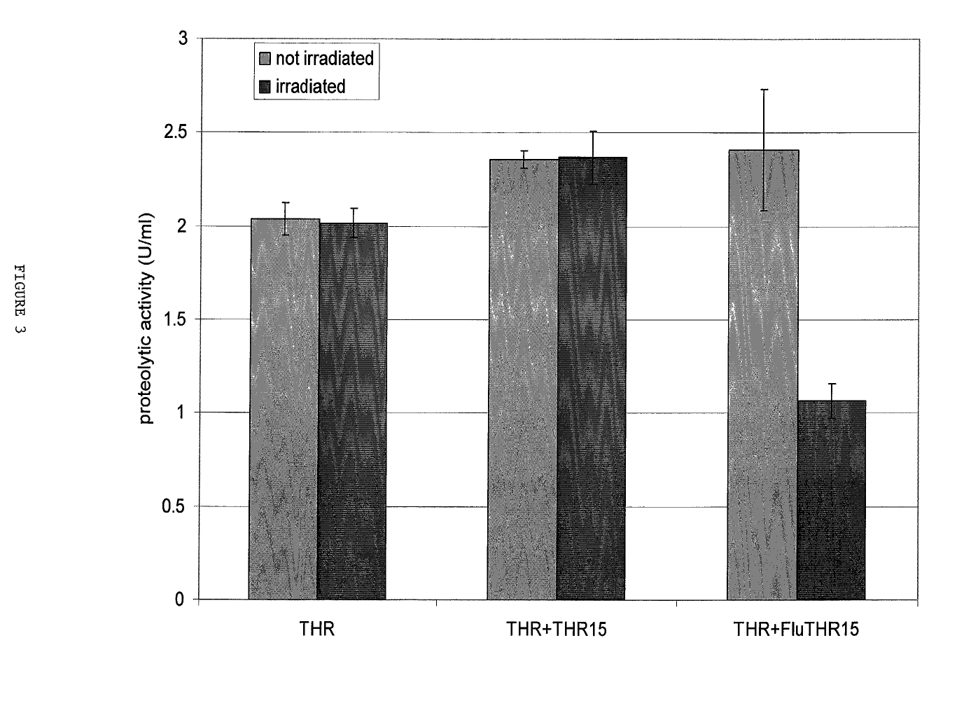

For CALI experiments, samples were irradiated in the microtiter plate using a tunable, multi-line Innova 90C (Coherent Inc., Santa Clara, Calif.) continuous wave argon ion laser set to 496 nm, with the power set to 800 mW. The beam diameter was adjusted to 5 mm with an interjected zoom beam expander to fit the well of a standard 96-well plate. Every well was irradiated for 30 seconds. The irradiated aliquots were pooled and activity measurements were done in triplicate for each sample. The extent of CALI effect (inactivation) was calculated as a percentage of the thrombin activity measured in a non-irradiated control. The results of the CALI experiments are shown in FIG. 3. The activity in U/ml is reported for thrombin alone, with specific aptamer THR15 and with fluorescein-labeled aptamer Flu-THR15. The latter showed a 55% thrombin inactivation upon irradiation (CALI effect).

The remaining sample (˜1 ml) was incubated with 6% trichloroacetic acid, followed by an 1 hour incubation on ice to precipitate the protein. The protein was centrifuged at 13K rpm and washed twice with cold acetone. Using standard protocols, the pellet was resuspended in 20 μl of 0.5 M Tris buffer, containing 6 M guanidinium chloride and incubated for 1 hour at 37° C. with 1 mM dithiotreithol to reduce the disulfide bonds. The solution was incubated for 45 minutes at room temperature in darkness with 5 mM iodoacetamide to alkylate the reduced cysteine residues. The reaction was stopped by adding an excess of 2-mercaptoethanol. The alkylated thrombin was then desalted and the light chain and the heavy chain were separated by reverse phase chromatography on selfpacked POROS 50 R2 (PerSeptive Biosystems) spin microcolumns (5-10 μl resin bed) . The solution was loaded onto the column, which was previously equilibrated with 5% formic acid. The bound species were washed with 30 μl 5% formic acid, then stepwise eluted with 3×10 μl of 35% acetonitrile/5% formic acid in water, followed by 3×10 μl of 70% acetonitrile/5% formic acid in water.

As a control experiment a non-irradiated thrombin sample was prepared according to the same protocol in order to obtain MS data of an untreated protein.

MS Analysis

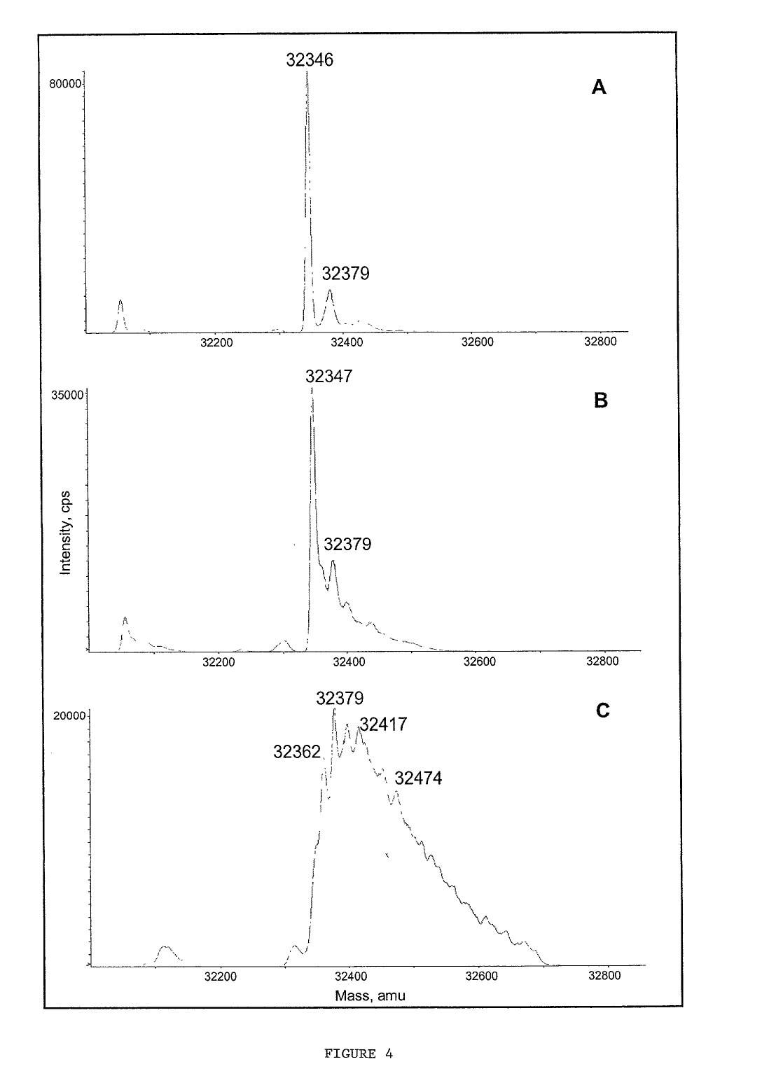

The two thrombin chains were separately analyzed by nano-electrospray on a QqTOF mass spectrometer (Q-STAR, Applied Biosystems). In order to determine the extent of covalent modification on the protein chains, the results which were obtained for the irradiated samples were compared with those of untreated thrombin, as a shown in FIG. 4.

Significant modification occurred at the heavy chain only. The heavy chain of both samples (CALI-modified thrombin and untreated thrombin) were further analyzed to determine the location of the modification(s) at the polypeptide chain. For this purpose, the samples derived from the 70% acetonitrile step elution (see above) were dried and resuspended in 20 μl of 50 mM ammonium bicarbonate buffer, pH 8 and treated alternatively with trypsin or endo-protease GluC in a 1:20 w/w ratio, for 16 hours at 37° C. Since it is known that thrombin is N-glycosylated, the peptide mixture was further incubated with N-Glycosidase F (Roche), in order to remove all N-glycans.

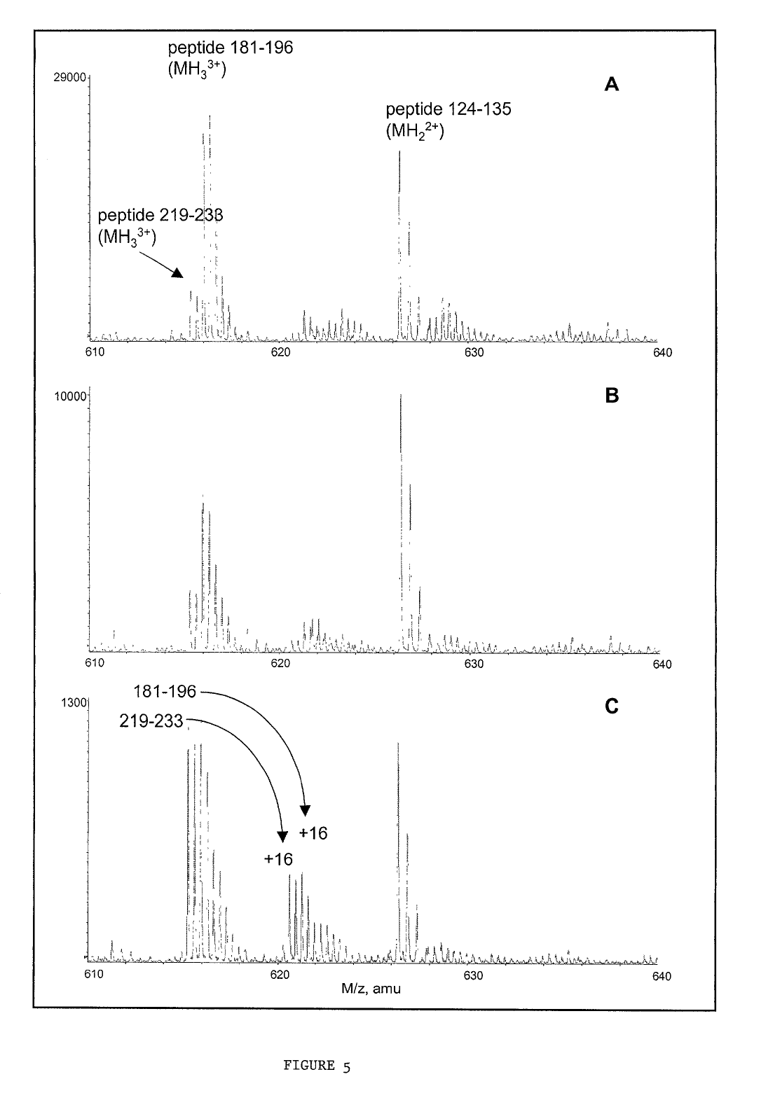

NanoES-MS analysis of the tryptic digests was performed preliminary for the untreated thrombin heavy chain in order to identify peaks in the spectra that correspond to specific tryptic or GluC peptides. Sample preparation for analysis included desalting of the peptide mixtures on self-packed reverse phase POROS R2 microcolumns, followed by a 2-step elution with 20% acetonitrile/5% formic acid in water, followed by 70% acetonitrile/5% formic acid in water; the two fractions were then analysed separately by nanoES-MS. By this This analysis allowed to obtain a protein coverage of 90% for the thrombin heavy chain. A similar procedure was carried out for the irradiated thrombin samples. FIG. 5 shows the nanoES-MS analysis of the peptide mixtures obtained by hydrolysis with trypsin of isolated thrombin heavy chain. Spectra were recorded for each sample in the range m/z 400-2000. FIG. 5 shows only a representative range of the entire spectrum. The corresponding spectra of the different treated samples were compared in order to detect modified peptides. The irradiated thrombin samples showed a total of 5 tryptic peptides with a mass shift of +16, as shown in FIG. 5 for two representative peptides. The observed mass shift could be due to amino acid side chain oxidation.

We claim:

1. A method for identifying one or more functional sites in a protein, the method comprising:
a) contacting the target protein with a binding partner linked to a laser-activatable marker (tag) (BP-tag), to form a complex of target protein and BP-tag;
b) irradiating the complex of target protein and BP-tag with laser light to generate free radicals which selectively alter the bound target protein at the binding site; and
c) identifying the selectively altered region of the protein by a combination of protein cleavage and mass spectrometry.

2. The method according to claim 1, further comprising identifying the selectively altered region of the protein by de novo sequencing.

3. The method according to claim 1, characterized in that the binding partner for the protein is selected from the group consisting of dsFv, scfv, Fab, diabody, immunoglobulin-like molecules, peptides, RNA, DNA, PNA, and small organic molecules.

4. The method according to claim 3, characterized in that the binding partner is derived from a combinatorial library.

5. The method according to claim 1, characterized in that the laser-activatable marker is a substance which can be bound to a protein and which generates free radicals on irradiation with laser light.

6. The method according to claim 5, characterized in that the laser-activatable marker is selected from the group consisting 4′,5′-dichloro-2′,7′-dimethoxy-fluorescein, fluorescein isothiocyanate, eosin, erythrosine, hydroxycoumarin, malachite green, malachite green isothiocyanate, methoxycoumarin, naphthofluorescein, 2′,4′,5′,7′-tetrabromosulpho-fluorescein and tetramethylrhodamine.

7. The method according to claim 6, characterized in that the laser-activatable marker is malachite green isothiocyanate or fluorescein isothiocyanate.

8. The method according to claim 1, characterized in that the protein cleavage is carried out chemically or enzymatically.

9. An apparatus characterized in that it is an automated system of integrated independent units/parts which can be used for identifying functional sites in proteins and which comprises:
a) an automated LBP screening machine for generating specific LBPs which are directed against specific target molecules or ligands;
b) a chromophore synthesizing apparatus for producing chromophores;
c) an LBP-chromophore coupling apparatus for linking the selected LBPs and the synthesized chromophores;
d) a loading apparatus for transferring the LBP-tag into predetermined wells which are coated with the target molecule or target ligand in the assay platform;
e) a transfer robot arm for moving the assay platform into the laser system;
f) a sample transfer robot for moving the samples into an LBP-tag/ligand separating apparatus;
g) a protein cleaving apparatus;
h) a mass spectrometer;
i) a database; and
j) a central computer system.
